(19) United States
(12) Patent Application Publication
(10) Pub. No.: US 2026/0245724 A1
(43) Pub. Date: Aug. 20, 2026

(54) NEURAL TEMPORAL FINGERPRINTING AND META-LEARNING FOR DISEASE DETECTION

(71) Applicants: SHUBHAM CHANDRA, Katy, TX (US); Gaurav Chandra, Dallas, TX (US); Kirsten Bischof, Dallas, TX (US)

(72) Inventors: SHUBHAM CHANDRA, Katy, TX (US); Gaurav Chandra, Dallas, TX (US); Kirsten Bischof, Dallas, TX (US)

(21) Appl. No.: 19/054,617

(22) Filed: Feb. 14, 2025

(51) Int. Cl. G16H 50/20; A61B 5/00; A61B 5/346; A61B 5/372

(52) U.S. Cl. CPC G16H 50/20; A61B 5/0002; A61B 5/346; A61B 5/372; A61B 5/6814; A61B 5/6823; A61B 5/6828; A61B 5/6846; A61B 5/7203; A61B 5/7267

(57) ABSTRACT

This invention integrates Neural Temporal Fingerprinting (NTF) with meta-learning to create a dynamic, adaptive system for detecting neurological disorders. By combining CNNs for spectral analysis, GraphSAGE for functional connectivity, and MAML for few-shot learning, the system identifies complex spatiotemporal EEG patterns, enabling accurate detection of Alzheimer's, PTSD, epilepsy, dementia, and comatose states. The system processes real-time or stored EEG data, extracting temporal and spatial biomarkers, while meta-learning rapidly adapts to new cases with minimal retraining. A reward-penalty mechanism optimizes classification, prioritizing critical markers while reducing errors. Outputs include risk scores and clinician-friendly insights, supporting real-time monitoring via wearables, clinical EEG systems, and telemedicine. This scalable AI-driven framework enhances neurological diagnostics, mental health monitoring, neurorehabilitation, and brain-computer interfaces (BCIs), offering a transformative approach to cognitive and neurological health assessment.

### BACKGROUND TECHNICAL FIELD

The present disclosure pertains to a method and system for predicting outcomes using Electroencephalography (EEG) and Electrocardiogramata. This approach integrates Neural Temporal Fingerprinting (NTF), a novel machine-learning technique employing Model-Agnostic Meta-Learning (MAML) and Graph Neural Networks (GNNs). By analyzing temporal EEG and ECG patterns, the system provides accurate neurological condition predictions, offering valuable insights for healthcare, diagnostics, and long-term monitoring. No federally/Government-sponsored research or development-based funding was used in this research.

This is a CIP of patent application Ser. No. 18/979,873.

### BACKGROUND OF THE INVENTION

Traditional biomedical signal processing methods rely on static models with pre-defined rules for detecting anomalies. These approaches often fail to adapt to inter-patient variability and dynamic physiological changes. EEG and ECG signals are highly sensitive to environmental noise, making it challenging to develop a robust diagnostic framework. The invention addresses these challenges by introducing a graph-based meta-learning approach that treats electrodes as nodes and their relationships as edges, allowing for an adaptable and self-improving predictive system

Electroencephalography (EEG) and Electrocardiogramare vital non-invasive diagnostic tools that provide real-time monitoring of brain and heart activity. Their integration with AI-powered Edge computing and Graph Neural Networks (GNNs) significantly enhances the ability to detect, predict, and analyze complex neural and cardiovascular patterns, enabling faster, more precise, and adaptive health assessments.

EEG captures neuronal electrical activity through scalp electrodes, allowing the detection of various neurological disorders and cognitive functions. It plays a crucial role in diagnosing epilepsy, where abnormal brainwave patterns indicate seizure activity, and in Alzheimer's disease, where changes in EEG coherence and power spectra correlate with cognitive decline. EEG is also widely used in sleep disorder analysis, helping identify conditions such as insomnia, narcolepsy, and REM sleep behavior disorder. Beyond diagnostics, EEG is instrumental in neurofeedback therapy, providing real-time biofeedback for mental health applications like stress reduction and PTSD management. Additionally, EEG enables brain-computer interface (BCI) applications, which facilitate direct neural interaction with machines, revolutionizing assistive technology for individuals with disabilities.

ECG, on the other hand, records the electrical activity of the heart through chest and limb electrodes, making it essential for cardiovascular health assessment. It is widely used for diagnosing arrhythmias, where irregular heartbeats can indicate life-threatening conditions such as atrial fibrillation or ventricular tachycardia. ECG plays a crucial role in the early detection of heart attacks, assessing myocardial infarctions by identifying electrical disturbances in the heart muscle. Moreover, ECG enables continuous monitoring of heart rate variability (HRV), which is essential for understanding stress responses, autonomic nervous system function, and chronic disease progression. The integration of ECG in wearable devices and telemedicine platforms has further expanded its utility, enabling remote patient monitoring and real-time cardiovascular insights without requiring in-hospital assessments. Together, EEG and ECG provide complementary neural and cardiac data, offering a holistic approach to early diagnosis, personalized treatment, and improved health outcomes. Their integration with artificial intelligence (AI) and predictive analytics enhances early disease detection, continuous monitoring, and precise diagnostics, making them indispensable tools for wearables, IoT-based healthcare solutions, and telemedicine applications. By embedding EEG-ECG analysis into Edge AI devices, these technologies enable real-time, high-precision monitoring, improving both neurological and cardiovascular healthcare while ensuring faster and more efficient interventions.

Electroencephalography (EEG) plays a pivotal role in neurofeedback therapy, brain-computer interface (BCI) research, and cognitive neuroscience, offering valuable insights into brain function, neurological disorders, and real-time neural control systems. Neurofeedback training, which utilizes EEG signals, allows individuals to self-regulate brain activity, aiding in the treatment of ADHD, PTSD, anxiety, depression, and other neuropsychiatric conditions. By providing real-time visual or auditory feedback, neurofeedback helps patients modulate abnormal brainwave patterns, promoting cognitive stability and emotional regulation.

In brain-computer interface (BCI) applications, EEG serves as a non-invasive method for decoding neural activity, enabling direct communication and control for individuals with physical disabilities, locked-in syndrome, or spinal cord injuries. AI-driven machine learning algorithms interpret EEG signals in real time, facilitating thought-controlled prosthetics, assistive communication devices, and adaptive neurotechnologies. Additionally, EEG is extensively used in cognitive neuroscience research, studying brain function during tasks such as memory recall, problem-solving, decision-making, and emotional processing, contributing to a deeper understanding of neuroplasticity and cognitive adaptability.

The integration of EEG and ECG has become instrumental in the detection, monitoring, and management of PTSD and neurological diseases. EEG identifies brainwave abnormalities associated with PTSD, such as amygdala hyperactivity, prefrontal cortex underactivity, and dysfunctional theta-beta ratios, which correlate with heightened stress responses and impaired emotional regulation. These insights are used to develop targeted neurofeedback therapies, enabling patients to train their brains to restore normal neural activity. Complementing EEG, ECG measures heart rate variability (HRV), a critical marker of autonomic nervous system function, stress levels, and emotional resilience. The combination of brain and heart data provides a holistic approach to PTSD assessment, identifying biomarkers that link emotional dysregulation with physiological stress responses.

Beyond PTSD, EEG plays a crucial role in diagnosing neurological diseases, detecting distinct electrophysiological patterns linked to specific disorders. For example, spike-and-wave discharges are indicative of epileptic seizures, while disrupted theta rhythms are a hallmark of Alzheimer's disease. In anxiety disorders, hyperactive beta waves reflect excessive cortical excitation, correlating with restlessness and hyperarousal symptoms. ECG further enhances diagnostics by identifying cardiac irregularities associated with neurological conditions, such as arrhythmias linked to autonomic dysfunction in Parkinson's disease or cardiac anomalies associated with stress-induced neurological deterioration.

From a therapeutic perspective, EEG is a powerful tool for tracking treatment efficacy, enabling clinicians to monitor changes in brainwave activity following neurofeedback, pharmacological interventions, or cognitive therapy. When integrated with ECG, these assessments extend beyond the brain to analyze the interconnected impacts of neurological treatments on cardiovascular function, ensuring a comprehensive evaluation of patient health. With the integration of artificial intelligence (AI) and predictive analytics, EEG and ECG enable advanced real-time monitoring, early disease detection, and predictive modeling of disease progression. AI-powered Edge AI systems and Graph Neural Networks (GNNs) enhance the precision, adaptability, and efficiency of EEG-ECG analysis, allowing wearables, IoT-based monitoring systems, and telemedicine platforms to deliver faster, more accurate neurological and cardiovascular assessments. These innovations revolutionize neuroscience, improve diagnostics, and pave the way for innovative therapeutic strategies, fostering personalized, data-driven approaches to mental health and neurological care.

The 10-20 system is a widely used, standardized method for placing electrodes on the scalp during an electroencephalography (EEG) recording, ensuring consistency and reproducibility across individuals and research studies. Each electrode is named based on its location: frontal (F), central (C), parietal (P), occipital (O), temporal (T), and midline (Z for zero). Odd-numbered electrodes are positioned on the left hemisphere, while even-numbered ones are placed on the right hemisphere. The electrodes are strategically placed at 10% and 20% distances from anatomical landmarks such as the nasion (bridge of the nose) and inion (bony bump at the back of the head), ensuring uniform scalp coverage and accurate representation of underlying brain activity.

Scalp Electrodes and Their Role in Neurological Disorder Detection. EEG channels are strategically positioned to capture activity in different brain regions, providing critical insights into various neurological and psychiatric conditions.

Frontal Lobe (F3, F4, F7, F8, AF3, AF4): These channels are highly relevant for epilepsy, ADHD, and depression. Frontal seizures are commonly detected in these locations, while ADHD is characterized by altered frontal activity patterns. Depression often manifests as asymmetry in frontal lobe activity, particularly between the left and right hemispheres.

Central Region (C3, C4, Cz): The motor cortex, located in this region, is essential for movement control. Conditions such as Parkinson's disease and ALS (Amyotrophic Lateral Sclerosis) often exhibit abnormalities in these channels, reflecting impaired motor function. Temporal Lobes (T3, T4, T5, T6, F7, F8): These areas are crucial for memory processing and language comprehension. Temporal lobe epilepsy is a common disorder associated with abnormal activity in this region, while Alzheimer's disease often presents with early-stage temporal lobe dysfunction.

Parietal Lobe (P3, P4, Pz): The parietal region is responsible for cognitive functions, spatial awareness, and sensory integration. This area is commonly affected in Alzheimer's disease and dementia, where reduced parietal activity is a hallmark of cognitive decline. Occipital Lobe (O1, O2): This region is responsible for visual processing and is associated with visual epilepsy, migraine disorders, and occipital lobe seizures. EEG analysis of this region helps detect abnormal visual cortex activity in such conditions.

Midline Channels (Fz, Cz, Pz): These electrodes capture generalized brain activity and are crucial for detecting generalized seizure disorders and autism spectrum disorder (ASD)-related neural patterns.

Although specific EEG channels correlate with neurological disorders, advanced signal processing techniques such as spectrogram analysis, machine learning, and frequency-domain feature extraction improve the accuracy of detection. Modern EEG diagnostics combine spatial and frequency analysis to enhance insights into neurological function and disease progression.

While scalp electrodes provide non-invasive insights into brain function, implantable EEG electrodes (also known as intracranial EEG or iEEG) offer higher spatial resolution and improved signal fidelity, particularly for patients with epilepsy, brain injuries, and deep-brain disorders. These invasive electrodes are surgically placed in subdural (cortical surface) or depth (penetrating brain tissue) configurations and are commonly used in: Resective Epilepsy Surgery Planning: iEEG provides precise localization of seizure foci before surgical removal of epileptic brain tissue.

Neurostimulation Therapies: Implantable EEG electrodes are integrated with responsive neurostimulation (RNS) systems to detect and suppress abnormal brain activity in real time. Deep Brain Stimulation (DBS): iEEG enhances neuromodulation therapies for Parkinson's disease, OCD, and treatment-resistant depression, guiding electrode placement and stimulation protocols.

Continuous, Long-Term Neurological Monitoring: Implantable electrodes allow for weeks to months of brain activity recording, enabling detection of rare seizure events, cognitive fluctuations, and disease progression tracking.

The integration of AI-powered Edge computing with scalp and implantable EEG electrodes is transforming neurological diagnostics. Graph Neural Networks (GNNs), meta-learning algorithms, and real-time predictive modeling enhance the ability to detect, classify, and predict brain abnormalities with unparalleled precision. Additionally, hybrid EEG-ECG analysis further strengthens the understanding of brain-heart interactions, offering comprehensive insights into neurocardiological disorders such as stress-induced arrhythmias, PTSD, and autonomic dysfunctions.

By leveraging implantable and non-invasive EEG, machine learning-based analytics, and Edge AI-driven predictive models, modern neuroscience is advancing diagnostics, enhancing treatment personalization, and improving long-term neurological care.

Implantable EEG electrodes, also known as intracranial EEG (iEEG) electrodes, represent a breakthrough in neurological diagnostics and treatment by providing high-resolution, direct brain recordings that surpass the limitations of traditional scalp EEG electrodes. Unlike non-invasive EEG, which captures brain activity from the scalp, implantable electrodes are surgically placed either on the cortical surface (subdural electrodes) or deep within brain structures (depth electrodes) to record precise neural activity from targeted regions. These electrodes are widely used in epilepsy monitoring, neurostimulation therapies, brain-machine interfaces (BMIs), and neuropsychiatric disorder treatments.

Model-Agnostic Meta-Learning (MAML) is a few-shot learning framework designed to enable deep learning models to rapidly adapt to new tasks with minimal data. Unlike traditional training approaches that optimize models for a single task, MAML trains models to be highly adaptable, allowing them to generalize across multiple unseen datasets or conditions. It achieves this by learning an optimal initialization of parameters that can be fine-tuned with just a few gradient updates when exposed to new data. This approach is particularly beneficial in applications where labeled data is scarce, expensive, or highly variable, such as neurological disease detection, medical diagnostics, and real-time EEG analysis.

MAML operates by simulating meta-training and meta-testing phases, where the model is trained on a variety of small tasks and then tested on unseen but related tasks. Instead of optimizing the model for a specific problem, MAML optimizes for adaptability, ensuring that the model's parameters can quickly converge to new solutions with minimal updates. This makes MAML an ideal choice for personalized medicine, dynamic neural monitoring, and real-time anomaly detection, where traditional deep learning models would require extensive retraining. By leveraging this meta-learning approach, AI systems can become more responsive, efficient, and capable of generalizing across diverse patient populations and evolving neurological conditions.

The invention integrates Neural Temporal Fingerprinting (NTF) with Meta-Learning to revolutionize the detection and classification of neurological disorders. Unlike prior patents that employ static machine learning architectures and predefined loss functions, this invention leverages dynamic adaptation through GraphSAGE for spatial feature extraction, CNNs for spectral analysis, and MAML-based Meta-Learning for few-shot learning. Traditional models often struggle to generalize across diverse EEG datasets due to fixed optimization strategies. In contrast, our invention continuously adapts to new patient data with minimal retraining, significantly improving diagnostic accuracy for Alzheimer's, PTSD, epilepsy, dementia, and coma states.

Prior art solutions primarily rely on single-layer CNNs, Random Decision Forests, or other static classifiers, limiting their ability to capture complex temporal dependencies in EEG signals. These approaches are effective within predefined datasets but lack generalization to unseen neurological patterns, particularly in real-world clinical environments. Our invention addresses this gap by combining CNNs for temporal biomarker extraction with GraphSAGE for functional connectivity analysis, forming a spatiotemporal neural fingerprint of brain activity. This enables a more comprehensive understanding of neurological function, surpassing the capabilities of prior models that fail to integrate both spectral and network-level insights.

A key differentiation lies in the optimization strategy. Prior art solutions often rely on static loss functions, which do not dynamically adjust to new patient data or address imbalanced datasets common in neurological diagnostics. Our invention overcomes this limitation by incorporating Meta-Learning, which enables rapid adaptation to novel EEG patterns. This few-shot learning mechanism ensures that even with limited training samples, the system can quickly fine-tune its parameters, offering personalized neurological assessments with high sensitivity and specificity. Unlike conventional approaches that require frequent model retraining, our adaptive learning framework ensures real-time applicability, making it particularly suitable for wearable EEG monitoring, telemedicine, and remote diagnostics.

Furthermore, unlike prior patents that focus solely on either EEG or ECG analysis, our invention extends its applicability to cross-modality diagnostics, integrating EEG and ECG data for more holistic neurological and cardiovascular assessments. This enables the identification of conditions influenced by both neural and cardiovascular factors, such as stress disorders, sleep disturbances, and neurodegenerative diseases. By merging spectral, connectivity, and adaptive learning components, the invention enhances its diagnostic range beyond epilepsy and seizures, offering a scalable solution for a broad spectrum of neurological disorders.

Applications extend beyond static diagnostic models, making this invention particularly effective for real-time neurological monitoring, mental health assessments, and adaptive brain-computer interfaces (BCIs). Unlike prior patents, which struggle to scale across evolving datasets and diverse patient populations, this invention provides a dynamic, meta-learning-driven framework capable of self-updating without the need for extensive retraining. Its robust scalability and adaptability set a new benchmark in AI-driven bio-signal analysis, paving the way for next-generation clinical diagnostics and real-time neurological monitoring in both hospital and home-care settings.

### SUMMARY OF THE INVENTION

This integrates Neural Temporal Fingerprinting (NTF) with Meta-Learning to create an adaptive and scalable system for detecting and classifying neurological disorders. Unlike traditional models that rely on static architectures, this system dynamically learns and evolves using a hybrid approach that combines CNNs for spectral analysis, GraphSAGE for functional connectivity mapping, and MAML-based Meta-Learning for real-time adaptation. By processing EEG signals from both scalp (10-20 system) and implantable electrodes, it provides a comprehensive analysis of neural activity across surface and deep brain regions, enhancing the detection of Alzheimer's, PTSD, epilepsy, dementia, coma states, and other cognitive impairments.

The system constructs a spatiotemporal neural fingerprint unique to each patient's brain activity by leveraging CNNs to extract temporal biomarkers from EEG spectrograms and GraphSAGE to electrode connectivity. Unlike conventional models that rely on fixed architectures, single-layer CNNs, or decision trees, this hybrid framework enables multi-modal learning, capturing both frequency-based abnormalities and disruptions in functional connectivity. The inclusion of implantable electrodes further enhances precision, making it particularly beneficial for applications such as epilepsy surgery planning and deep brain stimulation assessments.

A major innovation of this invention is the integration of meta-learning, allowing the system to continuously adapt to evolving EEG patterns. Traditional models require large labeled datasets and frequent retraining, making them impractical for diverse patient populations and real-world clinical use. In contrast, the MAML-based learning module fine-tunes its parameters dynamically, enabling few-shot learning for rapid adaptation to new neurological patterns. This feature makes the system ideal for personalized medicine, supporting real-time monitoring in wearable EEG devices, hospital EEG systems, and telemedicine applications.

Another key advancement is the dynamic reward and penalty-based optimization framework, which ensures the model prioritizes critical neurological events-such as seizures, Alzheimer's progression, and REM sleep anomalies—while minimizing false positives. Unlike static loss functions used in prior models, this adaptive optimization enhances classification accuracy and reliability, making the system highly suited for high-stakes clinical applications. Furthermore, the integration of cross-modality data from ECG extends the system's capabilities, allowing a holistic assessment of conditions affecting both neural and cardiovascular functions, such as stress disorders, autonomic dysfunctions, and sleep disturbances.

With its scalable, self-updating architecture, this invention is designed for both clinical diagnostics and real-time neurological monitoring. It has broad applications in mental health assessments, brain-computer interfaces (BCIs), neurorehabilitation, and adaptive neural prosthetics. The ability to process data from both non-invasive and invasive EEG electrodes ensures high precision, making this a groundbreaking solution in AI-driven neurological diagnostics.

### DETAILED DESCRIPTION OF THE INVENTION

The invention integrates Neural Temporal Fingerprinting (NTF) with Meta-Learning, introducing an advanced AI-driven approach for neurological disease detection using EEG signals from both scalp electrodes (10-20 channels) and implantable electrodes. Unlike traditional methods that rely on static machine learning architectures, this invention employs a hybrid deep learning framework combining Convolutional Neural Networks (CNNs), GraphSAGE, and Model-Agnostic Meta-Learning (MAML) to construct a spatiotemporal neural fingerprint of brain activity. The system is designed to rapidly adapt to new neurological conditions with minimal training data, making it particularly effective for rare disorders, evolving disease states, and personalized medical diagnostics.

At its core, the system processes EEG spectrograms through a CNN-based feature extractor, which identifies frequency-domain abnormalities associated with conditions like Alzheimer's, epilepsy, PTSD, dementia, and coma states. The feature extraction function can be represented as:

$${X}_{c n n} = {f}_{c n n} ( {S}_{E E G} ) \qquad Equation 1$$

Where S_{EEG}) represents the spectrogram transformation of the EEG signal and X_{cnn} denoted the extracted temporal features. These features are then complemented by Graphsage which models EEG electrode connectivity as a Graph G=(V, E), where nodes V represent electrodes and edges E define functional relationships between them. The node embedding update follows:

$${h}_{υ}^{( k )} = σ ( {W}_{k} \cdot AGG ( {h}_{μ}^{( k - 1 )} : μϵ N ( ν ) ) \qquad Equation 2$$

### Where

$${h}_{υ}^{( k )}$$

is the feature representation of node v at layer k, and AGG(.) represents an aggregation function such as mean or max pooling. By integrating temporal and spatial EEG features, the system constructs a comprehensive neural signature, surpassing prior models that rely solely on one domain.
A key innovation of this invention is the incorporation of Model-Agnostic Meta-Learning (MAML), which optimizes the model for rapid adaptation to new neurological patterns with minimal retraining. Unlike conventional models that require extensive labeled data, MAML adjusts model parameters by leveraging second-order derivative-based curvature information, ensuring that the system efficiently converges to a new task-specific solution with limited examples. The meta-learning objective is defined as:

$${θ}^{*} = θ - α {∇}_{θ} \sum {ℒ}_{tas {k}_{i}} ( θ - β {∇}_{θ} {ℒ}_{tas {k}_{i}} ( θ ) \qquad Equation 3$$

where θ represents the model parameters, a is the learning rate for the meta-update, and β is the inner-loop adaptation step. The second-order gradient term [symbol] ensures that the model optimizes adaptability rather than performance on a single task. This curvature-based adjustment allows the model to rapidly detect and classify unknown neurological conditions with only a few patient samples.

Furthermore, a reward and penalty-based optimization mechanism enhances the system's classification accuracy. Traditional models suffer from imbalanced datasets and high false-positive rates, particularly in seizure detection, REM sleep analysis, and neurodegenerative disease classification. To address this, the model dynamically adjusts the loss function based on clinical significance using:

$$ℒ = {ℒ}_{b a s e} + γ \cdot ( {w}_{r e w a r d} \cdot TP - {w}_{p e n a l t y} \cdot FP ) \qquad Equation 3$$

where w_{reward} and w_{penalty} represent weight factors prioritizing true positives (TP) while penalizing false positives (FP). This adaptive weighting ensures higher sensitivity for critical neurological conditions, reducing misclassification errors and enhancing overall reliability.

Another distinguishing feature of this invention is its ability to integrate EEG and ECG data into a unified platform, expanding its scope beyond isolated neural conditions. Unlike prior methods that analyze either EEG or ECG separately, this system leverages cross-modal learning to detect conditions influenced by both neural and cardiovascular factors, such as autonomic dysfunction, stress-related disorders, and sleep abnormalities. The combined feature space is defined as:

$${X}_{multi} = {γ}_{1} {X}_{c n n} + {γ}_{2} {X}_{g r a p h} + {γ}_{3} {X}_{e c g} \qquad Equation 4$$

where γ₁, γ₂, γ₃ are adaptive weight factors balancing EEG spectral, connectivity, and ECG-derived features. This multi-modal approach enhances diagnostic precision and supports personalized neurophysiological assessments.

The system is designed for real-time clinical diagnostics and continuous monitoring, with support for wearable EEG devices, implantable electrodes, and telemedicine applications. Unlike prior patents that require offline data processing and retraining, this invention enables on-the-fly adaptation, making it ideal for neurological disease progression tracking, neurorehabilitation, and personalized treatment plans. Its scalable architecture and curvature-driven adaptability establish a new standard for AI-driven neurology, offering a breakthrough solution for detecting, classifying, and monitoring neurological disorders dynamically.

### Advantages of the Invention

This invention offers a transformative advancement in neurological disease detection by integrating Neural Temporal Fingerprinting (NTF) with Meta-Learning, creating a highly adaptive and scalable system. Unlike traditional machine learning models that require large datasets and frequent retraining, this system leverages second-order derivative-based curvature optimization to enable few-shot learning, allowing it to rapidly adapt to new neurological conditions with minimal labeled data. This feature is particularly beneficial for rare diseases, early-stage neurological disorders, and patient-specific diagnostics, significantly reducing training overhead and computational costs.

By combining CNNs for spectral analysis, GraphSAGE for functional connectivity modeling, and MAML for meta-learning, the invention provides a comprehensive spatiotemporal representation of brain activity. This hybrid approach enhances classification accuracy for Alzheimer's, epilepsy, PTSD, dementia, and coma states, outperforming conventional methods that rely solely on either frequency-based or network-based analysis. The inclusion of 10-20 standard EEG electrodes and implantable electrodes further increases diagnostic precision, making the system suitable for both non-invasive and invasive neural monitoring.
A key advantage of the invention is its reward and penalty-based optimization mechanism, which prioritizes critical neurological events while reducing false positives, ensuring high sensitivity and specificity in real-time clinical settings. Unlike static loss functions used in prior models, this adaptive weighting system dynamically adjusts classification priorities based on clinical relevance, making it more reliable for high-risk conditions such as seizure detection and REM sleep abnormalities. Additionally, the ability to integrate EEG and ECG data into a unified framework enhances cross-modality diagnostics, allowing for a holistic assessment of conditions influenced by both neural and cardiovascular factors, such as stress disorders, autonomic dysfunction, and neurodegenerative diseases.
Another significant advantage is the system's real-time adaptability. Unlike prior art that requires offline data analysis and periodic retraining, this invention continuously self-updates based on evolving EEG patterns, making it ideal for wearable EEG systems, telemedicine platforms, and long-term neurological monitoring. This ensures seamless deployment in both clinical and home-care settings, expanding its application to brain-computer interfaces (BCIs), mental health assessments, neurorehabilitation, and personalized medicine. By combining scalability, efficiency, and dynamic learning, this invention sets a new benchmark in AI-driven neurological diagnostics, offering a breakthrough solution for detecting, classifying, and monitoring complex brain disorders with unprecedented accuracy and adaptability.

### BRIEF DESCRIPTION OF THE DRAWINGS

FIG. 1 illustrates the different modules for NFT and Meta Learning for Neurological disease detection. Starting with data acquisition, 1 EEG and ECG signals are collected from electrodes (scalp or implantable). In preprocessing, 2, artifacts are removed, and the data is normalized to enhance quality. Next, feature extraction, 3 identifies critical patterns from the preprocessed signals. CNN analysis, 4 focuses on extracting spectral features to uncover temporal biomarkers, while connectivity modeling, 5 employs graph-based methods to analyze functional relationships between electrodes. Finally, the meta-learning module, 6 enables dynamic adaptation to new tasks and conditions using few-shot learning, ensuring robust and personalized neurological diagnostics. This pipeline provides a comprehensive, scalable solution for detecting and monitoring neurological disorders.

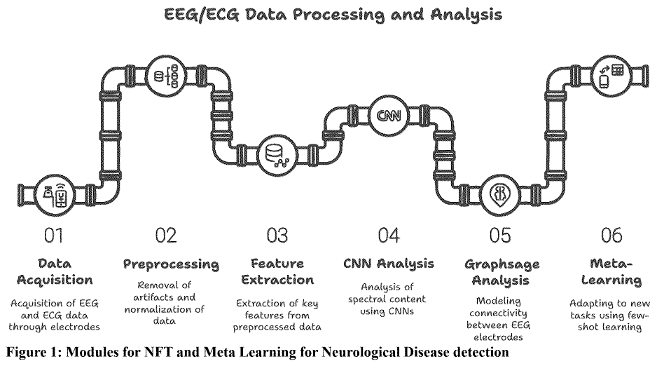

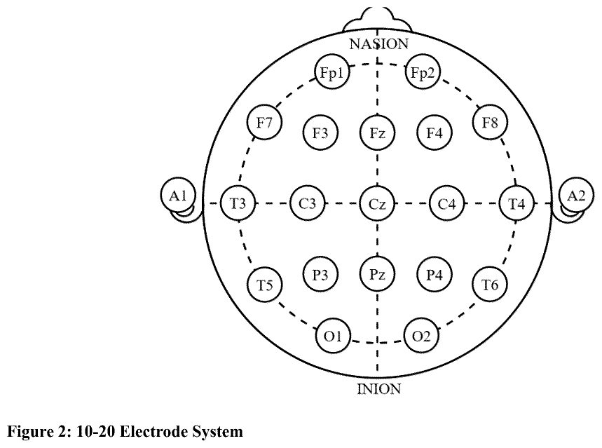

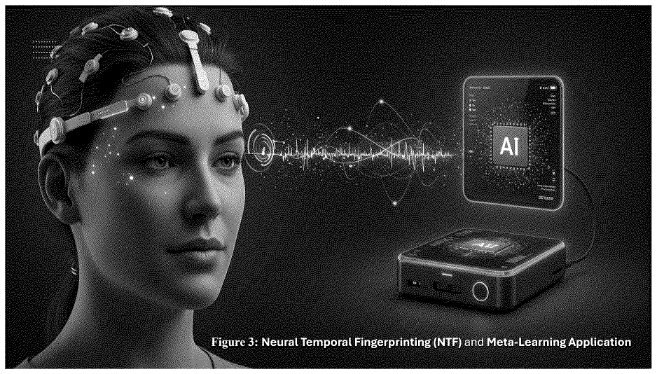

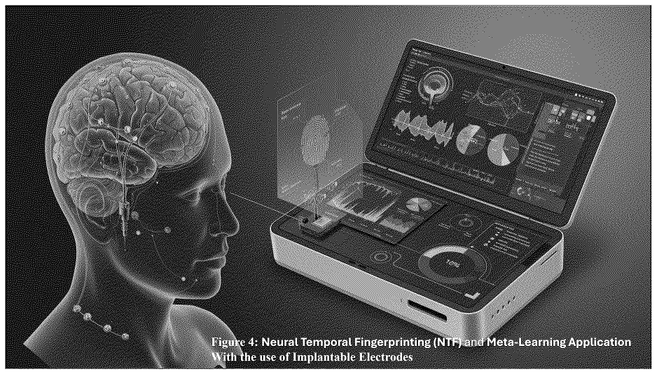

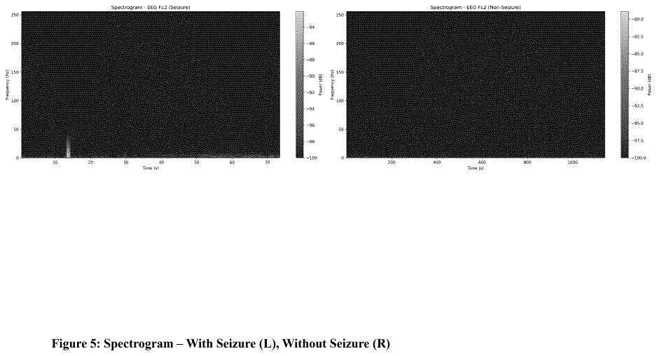

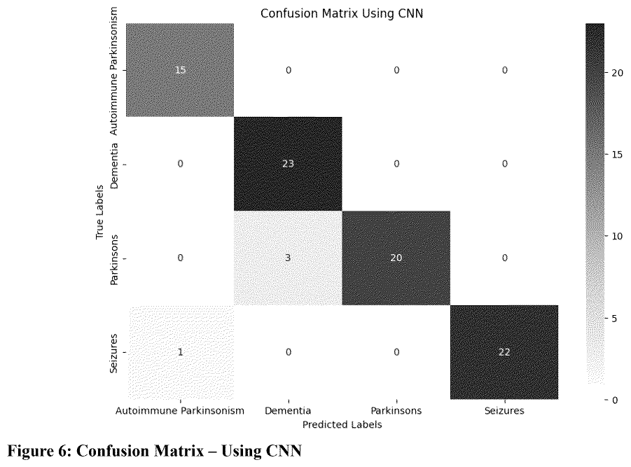

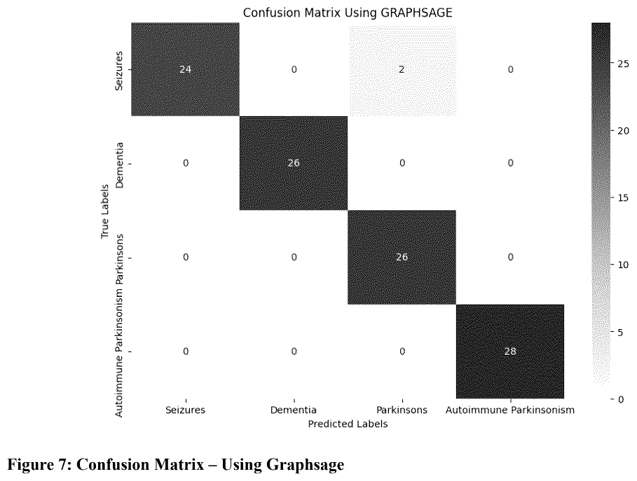

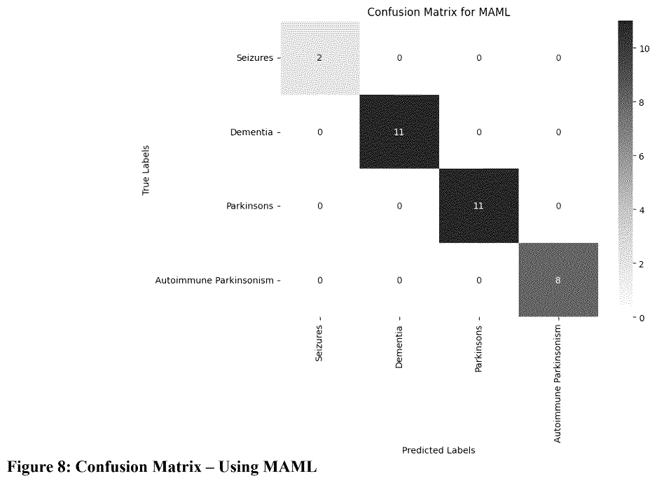

In FIG. 2, the 10-20 electrode system, which is an internationally recognized standard for placing electrodes on the scalp for electroencephalography (EEG) recordings. This system is designed to ensure consistent and reproducible electrode placement, facilitating reliable comparisons across studies and clinical applications. The name “10-20” refers to the proportional distances (10% or 20%) between electrodes, relative to the size of the head, ensuring coverage of all major brain regions. Key electrode positions correspond to specific anatomical landmarks such as the nasion (bridge of the nose) and inion (prominent point at the back of the skull).

Electrodes are labeled according to their location: Fp (frontal pole), F (frontal), C (central), T (temporal), P (parietal), and O (occipital). Odd numbers (e.g., Fp1, T3) represent the left hemisphere, even numbers (e.g., Fp2, T4) the right, and Z (e.g., Cz) indicates the midline. This system provides comprehensive spatial coverage for capturing neural activity patterns and is widely used in clinical diagnostics, brain-computer interface research, and neurophysiological studies.

FIG. 3 represents the application of Neural Temporal Fingerprinting (NTF) and Meta-Learning for advanced neurological disease detection. The individual is equipped with a high-tech EEG headset featuring multiple electrodes, which capture electrical brain activity across both surface and deep neural regions. The captured signals are processed in real-time by an AI-powered system, leveraging CNNs for extracting temporal biomarkers and GraphSAGE for mapping functional connectivity between electrodes.

The visualization of dynamic waveforms illustrates the neural signals being transformed into spatiotemporal neural fingerprints unique to the individual's brain activity. The AI processing unit integrates these fingerprints with a MAML-based Meta-Learning framework, enabling the system to quickly adapt to new neurological conditions with minimal retraining. This adaptability allows for the detection of complex disorders, including Alzheimer's, epilepsy, PTSD, and dementia.
The holographic interface highlights the system's ability to analyze EEG patterns and predict neurological disease progression in real-time. By incorporating both surface EEG data and implantable electrodes, the system achieves enhanced precision, supporting applications like epilepsy surgery planning and deep brain stimulation assessments. This figure underscores the potential of combining NTF and Meta-Learning to revolutionize personalized medicine, enabling real-time monitoring and diagnostics across diverse clinical environments.

FIG. 4 showcases a cutting-edge system that integrates Neural Temporal Fingerprinting (NTF), Meta-Learning, Edge AI, and implantable electrodes to revolutionize neurological disease detection and monitoring. The brain diagram illustrates the use of both implantable and surface electrodes for capturing high-resolution neural activity across surface and deep brain regions. These signals are processed to identify unique spatiotemporal neural fingerprints, enabling precise diagnosis of neurological disorders such as epilepsy, Alzheimer's, and PTSD. The inclusion of implantable electrodes provides enhanced precision by capturing intracranial neural data, critical for applications like epilepsy surgery planning and deep brain stimulation (DBS) optimization.

The system employs Meta-Learning to dynamically adapt to new neurological patterns with minimal retraining, accommodating diverse patient profiles and evolving conditions. This capability is complemented by Edge AI, as data processing occurs directly on the portable AI-powered device rather than relying on cloud computing. This ensures low latency, improved data privacy, and real-time analysis-key for time-sensitive scenarios such as seizure detection or DBS monitoring. The holographic visualization of neural fingerprints and detailed metrics, including EEG waveforms and connectivity maps, provides clinicians with actionable insights and supports personalized medical decisions.
Designed for real-time monitoring and personalized diagnostics, this system offers applications in wearable EEG devices, hospital EEG systems, and telemedicine platforms. By seamlessly combining advanced neural data acquisition, adaptive learning, and on-device computation, it sets a new standard for precision in neurological care and diagnostics.

In FIG. 5, the spectral differences between seizure and non-seizure events recorded from the electrode (FC2) using spectrogram analysis are documented.
1. Seizure Event (Left):
   The spectrogram demonstrates concentrated power at lower frequencies (below 50 Hz) during the seizure, indicated by the bright yellow and green areas. This reflects heightened neural activity typical during epileptic episodes, captured as distinct temporal and spectral patterns.
2. Non-Seizure Event (Right):
   The spectrogram shows low and evenly distributed power across the frequency range, characteristic of normal brain activity. The absence of high-intensity patterns indicates no significant neural disruptions.
   These visualizations underscore the system's ability to differentiate seizure and non-seizure events by identifying spatiotemporal neural fingerprints, a crucial feature for real-time epilepsy monitoring and diagnosis.

In FIG. 6, The confusion matrix presented illustrates the performance of a CNN-based classification model across four categories: Autoimmune Parkinsonism, Dementia, Parkinson's, and Seizures. The model demonstrates strong classification accuracy, as indicated by the high number of correctly classified instances along the diagonal of the matrix. Specifically, the model accurately predicts 15 instances of Autoimmune Parkinsonism, 23 instances of Dementia, 20 instances of Parkinson's, and 22 instances of Seizures. However, there are some misclassifications. Notably, three cases of Parkinson's are misclassified as Dementia, and one case of Seizures is misclassified as Autoimmune Parkinsonism. These misclassifications suggest that there may be overlapping feature representations between these conditions, which could require further fine-tuning of the model or enhancement of the training dataset to improve class differentiation. Overall, the CNN model exhibits high classification accuracy, particularly for Dementia and Seizures, with minimal misclassification errors. The results indicate that the model is effectively distinguishing between these neurological conditions, but some refinements may be necessary to further reduce the confusion in Parkinson's-related cases.

The FIG. 7 presents a Confusion Matrix for a classification model utilizing GraphSAGE to distinguish between four medical conditions: Seizures, Dementia, Parkinsons, and Autoimmune Parkinsonism. The matrix visually represents the performance of the model by comparing true labels (rows) with predicted labels (columns).

From the matrix, the model exhibits strong classification accuracy across most categories. Notably, the diagonal elements, which indicate correct predictions, show high values: 24 correctly classified Seizures, 26 Dementia cases, 26 Parkinsons cases, and 28 Autoimmune Parkinsonism cases. However, two Seizure cases were misclassified, suggesting minor misprediction in this category. The confusion matrix also highlights that there are no misclassifications among the Dementia, Parkinsons, and Autoimmune Parkinsonism categories, indicating that the model performs exceptionally well in distinguishing these conditions. The color intensity in the heatmap reinforces these observations, with darker shades representing higher classification counts. Overall, the GraphSAGE-based classification model demonstrates high accuracy, with minimal misclassifications. Future improvements could focus on enhancing the differentiation of Seizures from other conditions to further optimize the model's predictive capabilities.

The FIG. 8 represents a Confusion Matrix for MAML (Model-Agnostic Meta-Learning), illustrating the model's classification performance across four medical conditions: Seizures, Dementia, Parkinsons, and Autoimmune Parkinsonism. The matrix compares the true labels (rows) with the predicted labels (columns) to visualize the accuracy and misclassifications. The MAML model demonstrates high precision in classifying Dementia (11 correct predictions), Parkinsons (11 correct predictions), and Autoimmune Parkinsonism (8 correct predictions), with no misclassification among these conditions.

### EXAMPLES

The invention will now be illustrated, but not limited, by reference to the specific embodiments described in the following examples.

### Example 1

The table below illustrates a sample dataset containing patient-related EEG, ECG, and CT scan records. Each entry represents a unique patient, identified by the ID column. The HCID column represents a hospital or clinic-specific identifier. The dataset captures demographic information, including Age (in years), Gender, Location, and Ethnicity.

For EEG-related data, the EEG.edf extract column specifies whether the EEG file is available, with additional details if extraction is partial. The EEG Report and EEG Extract columns indicate the availability of corresponding EEG reports and extracted data, respectively. The EEG Date column records the dates of EEG recordings.
Similarly, for ECG data, the ECG Report column denotes the availability of ECG reports, while the ECG Date column logs the respective ECG test dates. Some patients may have multiple ECG reports, as indicated in the provided dataset.
Regarding CT scans, the CT Scan Report column identifies whether a CT scan report is available, and the CT Report Extract column confirms if extracted information from the report is accessible. The CT Date column records the date(s) of CT scans.

This example demonstrates the methodology that integrates meta-learning and graph-based deep learning to enhance classification accuracy for EEG-related medical conditions. The approach utilizes Model-Agnostic Meta-Learning (MAML) to enable fast adaptation to new tasks with minimal data, ensuring that the model generalizes well to unseen EEG patterns. Additionally, GraphSAGE is employed to capture spatial and temporal dependencies across EEG electrode nodes, leveraging their structural relationships for improved feature extraction. The pipeline begins with unsupervised learning, where raw EEG signals are preprocessed into spectrogram representations and used to train a Convolutional Neural Network (CNN) for feature extraction. These CNN-generated embeddings serve as input for the GraphSAGE model, which constructs a graph with EEG electrodes as nodes and their features as edges. GraphSAGE aggregates neighborhood information to learn node representations, capturing relationships between EEG channels.

For classification, MAML fine-tunes the learned representations by optimizing for quick adaptation to new datasets. The model is trained on a diverse set of EEG conditions, such as Seizures, Dementia, Parkinson's, and Autoimmune Parkinsonism, using a few-shot learning paradigm. The confusion matrix and classification report evaluate model performance, highlighting high accuracy across most classes, though some seizure cases require further refinement.
By combining CNN-based feature extraction, GraphSAGE's graph-based learning, and MAML's meta-learning capabilities, this methodology achieves a robust classification framework for EEG-based medical diagnosis. The model effectively generalizes across different neurological conditions, demonstrating the potential of hybrid deep learning architectures in medical AI applications.

TABLE 1
EXAMPLE TEST DATA

| | | Age (In | | | |
|---|---|---|---|---|---|
| ID | HCID | years) | Gender | Location | Ethinicity |
| E705A0D9 | Z4X9 | 34 | Female | North India | Indian |
| 9E610069 | Z4X9 | 77 | Female | North India | Indian |
| D714E364 | Z4X9 | 74 | Female | North India | Indian |
| C751F43F | A7B3 | 70 | Male | South India | Indian |

In Table 2, The classification performance of the CNN model is evaluated using precision, recall, and F1-score across four categories: Autoimmune Parkinsonism, Dementia, Parkinson's, and Seizures. The model demonstrates high precision and recall across all categories, with an overall accuracy of 95% on the test dataset of 84 samples.
For Autoimmune Parkinsonism, the model achieves a precision of 0.94, a recall of 1.00, and an F1-score of 0.97, indicating that all actual cases were correctly classified, with minimal false positives. Similarly, Dementia is classified with a precision of 0.88, a recall of 1.00, and an F1-score of 0.94, showing that while all cases were identified correctly, a few false positives may have been included.
Parkinson's classification achieves a perfect precision of 1.00, meaning that all predicted Parkinson's cases were correct. However, the recall is 0.87, suggesting that some actual Parkinson's cases were misclassified into other categories, resulting in an F1-score of 0.93. For Seizures, the model performs exceptionally well with both precision and recall close to 1.00 (1.00 and 0.96, respectively), yielding a high F1-score of 0.98. The macro average scores for precision, recall, and F1-score are 0.96, 0.96, and 0.95, respectively, indicating that the model performs well across all classes without being biased towards any particular one. The weighted average, which takes class support into account, also remains at 0.96 for precision and 0.95 for recall and F1-score, confirming a strong overall classification performance.
These results suggest that while the model is highly effective in classifying neurological conditions, slight improvements in distinguishing Parkinson's cases could further enhance its robustness.

TABLE 2
CLASSIFICATION REPORT FOR CNN

| | precision | recall | f1-score | support |
|---|---|---|---|---|
| Autoimmune Parkinsonism | 0.94 | 1.00 | 0.97 | 15 |
| Dementia | 0.88 | 1.00 | 0.94 | 23 |
| Parkinsons | 1.00 | 0.87 | 0.93 | 23 |
| Seizures | 1.00 | 0.96 | 0.98 | 23 |
| accuracy | | | 0.95 | 84 |
| macro avg | 0.96 | 0.96 | 0.95 | 84 |
| weighted avg | 0.96 | 0.95 | 0.95 | 84 |

In Table 3, The classification performance of the GraphSAGE model is evaluated based on precision, recall, and F1-score across four categories: Seizures, Dementia, Parkinson's, and Autoimmune Parkinsonism. The model exhibits excellent classification capabilities, achieving an overall accuracy of 98% on a test dataset of 106 samples.
For Seizures, the model attains a precision of 1.00, indicating that all predicted seizure cases were correct. However, the recall is 0.92, meaning some actual seizure cases were misclassified, resulting in an F1-score of 0.96. In the case of Dementia, the model performs perfectly with a precision, recall, and F1-score of 1.00, suggesting that all dementia cases were accurately classified with no false positives or negatives.
The classification performance for Parkinson's is also high, with a precision of 0.93 and a recall of 1.00, leading to an F1-score of 0.96. This indicates that while all actual Parkinson's cases were correctly identified, a small percentage of the predicted Parkinson's cases may belong to other categories. Similarly, Autoimmune Parkinsonism is classified flawlessly, achieving a precision, recall, and F1-score of 1.00, reflecting perfect identification without any misclassifications.
The macro average precision, recall, and F1-score all stand at 0.98, confirming that the model maintains a consistently high performance across all categories without bias toward any particular class. The weighted average results also match at 0.98, further reinforcing the model's robustness.
Overall, the GraphSAGE model demonstrates highly accurate classification, with near-perfect performance for Dementia and Autoimmune Parkinsonism, and minimal misclassification in Seizures and Parkinson's

TABLE 3
CLASSIFICATION REPORT FOR GRAPHSAGE

| | precision | recall | f1-score | support |
|---|---|---|---|---|
| Seizures | 1.00 | 0.92 | 0.96 | 26 |
| Dementia | 1.00 | 1.00 | 1.00 | 26 |
| Parkinsons | 0.93 | 1.00 | 0.96 | 26 |
| Autoimmune Parkinsonism | 1.00 | 1.00 | 1.00 | 28 |
| accuracy | | | 0.98 | 106 |
| macro avg | 0.98 | 0.98 | 0.98 | 106 |
| weighted avg | 0.98 | 0.98 | 0.98 | 106 |

In Table 4, the classification performance of the MAML model highlights its ability to achieve perfect accuracy (100%) even with a limited dataset. The model effectively classifies all four categories—Seizures, Dementia, Parkinson's, and Autoimmune Parkinsonism—with 1.00 precision, recall, and F1-score, demonstrating its strong adaptation capabilities despite a small number of training samples. The dataset consists of only 32 total samples, with Seizures having just 2 cases, Dementia and Parkinson's having 11 cases each, and Autoimmune Parkinsonism having 8 cases. Despite this small sample size, the model correctly classifies every instance, indicating its ability to generalize well with minimal training data. The macro and weighted averages all remain at 1.00, further reinforcing the model's robustness in few-shot learning scenarios.
These results underscore the power of MAML in handling low-data environments, making it particularly useful for medical applications where large labeled datasets may not always be available

TABLE 4
CLASSIFICATION REPORT FOR MAML

| | precision | recall | f1-score | support |
|---|---|---|---|---|
| Seizures | 1.00 | 1.00 | 1.00 | 2 |
| Dementia | 1.00 | 1.00 | 1.00 | 11 |
| Parkinsons | 1.00 | 1.00 | 1.00 | 11 |
| Autoimmune | 1.00 | 1.00 | 1.00 | 8 |

| | | | | | |
|---|---|---|---|---|---|
| accuracy | | | | 1.00 | 32 |
| macro avg | 1.00 | 1.00 | 1.00 | 32 | |
| weighted avg | 1.00 | 1.00 | 1.00 | 32 | |

### Example Explanation

This workflow is a practical example of how seizure-related datasets can be processed and analyzed using machine learning. The method demonstrates combining data engineering (file processing) with advanced AI techniques (CNNs) to create a robust predictive system for healthcare applications.

This example showcases the exceptional adaptability of the invention, achieving 100% classification accuracy for Dementia, Parkinson's, and Autoimmune Parkinsonism. Its few-shot learning capability enables it to generalize effectively in scenarios with limited training data, making it highly suitable for personalized diagnostics and real-time applications. Furthermore, the invention's scalability allows seamless integration into wearable health monitoring systems and telemedicine solutions, ensuring efficient, real-time disease detection and adaptive medical insights across diverse healthcare settings.

### Commercial Applications

The integration of Neural Temporal Fingerprinting (NTF) with Meta-Learning presents significant commercial applications in neurology, cardiology, wearable health monitoring, telemedicine, and personalized medicine. This AI-driven diagnostic framework enables real-time, adaptive disease detection using EEG and ECG data, making it highly valuable across multiple industries.

In healthcare and diagnostics, this invention revolutionizes early disease detection for conditions such as epilepsy, Alzheimer's, Parkinson's, PTSD, and cardiovascular anomalies. Unlike traditional diagnostic tools that require large labeled datasets and frequent retraining, this system uses Model-Agnostic Meta-Learning (MAML) to rapidly adapt to new neurological conditions with minimal data. This capability makes it particularly useful for hospitals, research labs, and clinical applications where patient variability is high. Furthermore, neurological rehabilitation centers can integrate this technology into real-time EEG monitoring systems to track treatment efficacy and adapt therapies dynamically.

In the wearable technology industry, the invention enhances smart EEG and ECG-enabled devices for continuous patient monitoring. The integration of Edge AI and Graph Neural Networks (GNNs) allows for real-time data processing on portable devices, eliminating the need for cloud-dependent analysis. This makes the technology well-suited for smartwatches, headbands, and biosensors, enabling consumers to track their neurological and cardiovascular health without needing frequent hospital visits. Companies developing next-generation health wearables and IoT-based remote monitoring solutions can leverage this system to provide personalized health insights, particularly for individuals at risk of stroke, seizures, and neurodegenerative diseases. The invention also has substantial applications in telemedicine and remote diagnostics. By integrating AI-powered EEG-ECG analytics, this system enables real-time neurological monitoring for patients in remote areas. Telehealth platforms can incorporate this technology to offer AI-driven consultations, providing neurologists and cardiologists with real-time insights into patient conditions without requiring in-person visits. This enhances early disease detection, reduces hospital burdens, and improves healthcare accessibility in underserved regions.

In mental health and neurofeedback therapy, this system can be applied to stress monitoring, PTSD therapy, and brain-computer interface (BCI) applications. The ability to detect cognitive states in real time makes it an excellent tool for personalized mental health interventions, such as adaptive meditation programs, anxiety monitoring, and EEG-based therapy tracking. Neurofeedback clinics and mental health applications can integrate this model to enhance patient engagement and optimize therapeutic outcomes.
The brain-computer interface (BCI) industry can also benefit significantly from this invention. The ability to extract spatiotemporal neural fingerprints using EEG and GNN-based analysis enhances human-computer interaction (HCl) technologies, enabling thought-controlled prosthetics, assistive communication devices, and adaptive neurotechnologies for individuals with paralysis, locked-in syndrome, or spinal cord injuries. This invention can significantly improve BCI-based assistive technologies, driving innovation in rehabilitation robotics and smart prosthetics.
From a pharmaceutical and clinical research perspective, the system enables personalized drug testing and treatment response analysis by monitoring EEG and ECG biomarkers over time. Pharmaceutical companies developing drugs for neurological and cardiovascular conditions can use this technology to measure the effectiveness of medications and adapt treatment strategies dynamically. The real-time adaptation capabilities of MAML ensure that the system can detect subtle neurological changes, allowing clinicians and researchers to assess disease progression more accurately.
In summary, this AI-driven diagnostic framework has broad commercial potential in healthcare, wearables, telemedicine, neurofeedback, BCI research, and pharmaceutical industries. By combining EEG, ECG, deep learning, and adaptive meta-learning, this invention offers a scalable, real-time, and highly precise approach to neurological and cardiovascular disease detection, shaping the future of personalized medicine and intelligent health monitoring solution.

1. A computerized system for disease prediction using electroencephalography (EEG) and electrocardiography (ECG) data, comprising:
A signal acquisition module configured to collect EEG data from scalp or implantable electrodes and ECG data from chest or limb electrodes;
A preprocessing unit that filters noise, normalizes signal values, and extracts features from EEG and ECG signals;
A deep learning framework integrating neural networks for spectral and connectivity-based analysis;
A meta-learning module that enables rapid adaptation to new patient data using few-shot learning techniques;
A predictive inference engine that fine-tunes classification parameters in real-time for detecting neurological and cardiovascular conditions;
An adaptive learning mechanism that updates predictive models based on newly acquired EEG-ECG signals, enabling personalized and evolving disease monitoring.

2. A computerized method for disease detection using EEG and ECG data, comprising:
Receiving EEG signals from scalp or implantable electrodes and ECG signals from chest or limb electrodes;
Preprocessing the signals by filtering noise, normalizing values, and segmenting data into time windows;
Extracting spectral and connectivity-based features using neural networks;
Applying meta-learning to train a classification model that adapts to new conditions with minimal labeled data;
Predicting the onset of neurological and cardiovascular diseases based on real-time neural fingerprinting;
Fine-tuning classification models dynamically using few-shot learning to continuously adapt to new patient data without extensive retraining.

3. An adaptive learning system for disease monitoring, comprising:
A hybrid deep learning architecture integrating spectral, connectivity, and meta-learning-based analysis;
A data acquisition module configured to capture EEG signals from scalp or implantable electrodes and ECG waveforms from wearable or medical-grade chest electrodes;
A real-time signal processing unit that extracts neural fingerprints from EEG and ECG patterns;
A learning engine that classifies medical conditions without requiring extensive labeled training datasets;
An AI-powered inference module that detects and predicts disease onset in real-time;
A telemedicine integration module that enables remote monitoring for tracking disease progression.

4. The system of claim 1, wherein a neural network models EEG electrode connectivity as a graph to represent functional relationships between neural regions, enabling improved disease classification.

5. The method of claim 2, wherein the meta-learning module dynamically updates patient-specific neural fingerprints by leveraging second-order optimization techniques to enhance adaptation to evolving disease states.

6. The system of claim 3, wherein the adaptive learning mechanism prioritizes critical neurological and cardiovascular events using an optimization framework to reduce false positives while maintaining high classification accuracy.
